(19) United States
(12) Patent Application Publication
(10) Pub. No.: US 2026/0098282 A1
(43) Pub. Date: Apr. 9, 2026

(54) CONVERSION OF CARBON DIOXIDE TO HIGHER VALUE CHEMICALS

(71) Applicant: The Texas A&M University System, College Station, TX (US)

(72) Inventors: Peng Zhang, College Station, TX (US); Kainan Chen, College Station, TX (US); Joshua Shuhua Yuan, College Station, TX (US); Susie Y. Dai, College Station, TX (US)

(21) Appl. No.: 19/114,447

(22) PCT Filed: Sep. 23, 2023
(86) PCT No.: PCT/US2023/074976

§ 371 Date: Mar. 22, 2025

Related U.S. Application Data
(60) Provisional application No. 63/409,548, filed on Sep. 23, 2022.

(51) Int. Cl. C12P 7/6409; C12N 9/02; C12N 9/10; C12P 7/6463; C12P 7/6481; C25B 3/03; C25B 3/07; C25B 3/26; C25B 11/031; C25B 11/054; C25B 11/075; C25B 11/089; C25B 15/08

(52) U.S. Cl. CPC C12P 7/6409; C12N 9/0008; C12N 9/0036; C12N 9/1029; C12P 7/6463; C12P 7/6481; C12Y 102/0101; C12Y 106/01001; C12Y 203/01086; C25B 3/03; C25B 3/07; C25B 3/26; C25B 11/031; C25B 11/054; C25B 11/075; C25B 11/089; C25B 15/081

(57) ABSTRACT

A method for the production of value added chemicals comprising: introducing carbon dioxide to a system comprising an electrochemical cell comprising a carbon dioxide reducing catalyst and an electrolyte under conditions suitable for conversion of carbon dioxide to one or more C₂ compounds; introducing at least a portion of the one or more C₂ compounds to a second system comprising a microbe and a media under conditions suitable for conversion of the C₂ compounds to one or more C₂₊ₙ compounds where n is from about 1 to about 100. A method of preparing value added chemicals comprising: a means for converting carbon dioxide to one or more C₂ compounds and a microbe wherein the C₂ compound is contacted with the microbe under conditions suitable for the formation of one or more C₂₊ₙ compounds.

### CROSS-REFERENCE TO RELATED APPLICATIONS

This application is a 35 U.S.C. § 371 national stage application of PCT/US2023/074976 filed Sep. 23, 2023, and entitled “Conversion of Carbon Dioxide to Higher Value Chemicals,” which claims priority to U.S. Provisional Application No. 63/409,548 filed Sep. 23, 2022 entitled “Compositions and Methods for Rapid Conversion of Carbon Dioxide to Higher Value Chemicals,” each of which is hereby incorporated herein by reference in its entirety for all purposes.

### STATEMENT REGARDING FEDERALLY SPONSORED RESEARCH OR DEVELOPMENT

Not applicable.

### TECHNICAL FIELD

The present disclosure relates generally to compositions and systems for use in carbon dioxide sequestration. More particularly, the present disclosure relates to compositions and coupled systems for the conversion of carbon dioxide to higher carbon number chemicals.

### BACKGROUND

The synthesis of fuels, chemicals, and materials from CO₂ is fundamental to human society. In the biosphere, plants and algae convert CO₂ through photosynthesis to macromolecules and diverse chemicals to sustain their own growth and feed other heterotrophic organisms. Most of the chemicals, materials, foods, and fuels routinely used in society are derived from photosynthesis, either directly (e.g. phototrophs) or indirectly (e.g. fossil fuels and biorefinery). However, the efficiency of photosynthesis is limited to about 1% in most terrestrial plants due to inefficient carbon conversion by RubisCO and the energy-extensive regeneration of carbon intermediates. RubisCO is an enzyme that catalyzes the first step of carbon fixation in the C3 pathway or Calvin cycle which leads to the formation of 2 molecules of 3-phosphoglyceric acid. Even though chemical synthesis has been reported for the successful conversion of CO₂ into selective compounds (e.g., urea) at the commercial scale, the scope of diverse chemical compounds obtainable from CO₂ conversion is still limited.

In consideration of the increasing CO₂ levels in the enviornment caused by human activities, alternative CO₂ conversion platforms are continuing to be developed. The goals of such platforms are generally twofold; carbon dioxide capture and commercial utilization of carbon dioxide. Achieving either goal would have a substantive societal import as mitigating the extent of global climate change and/or the manufacture of value added chemicals or precursors thereof from carbon dioxide would result in a substantive change in global economics.

One such carbon conversion platform involving integration of the catalytic CO₂ reduction with bioconversion could provide a carbon dioxide conversion platform meeting the aforementioned goals. However, the state-of-the-art technologies that could be used to construct such a platform are limited by inefficient electron and mass transfers, unfavorable metabolic kinetics, and inadequate molecular building blocks. Thus, an ongoing need exists for the identification and utilization of compositions, systems and methods for the capture and conversion of carbon dioxide to value added chemicals.

### BRIEF DESCRIPTION OF THE DRAWINGS

For a detailed description of the aspects of the disclosed processes and systems, reference will now be made to the accompanying drawings in which:

FIG. 1 is a schematic depiction of a carbon dioxide capture and conversion system.

FIG. 2 is a bar graph depicting the results of a comparison of theoretical fatty acid yields from either ethanol, acetate or glucose as the sole carbon source for Rhodococcus jostii RHA1.

FIG. 3A is a graph of the stimulated fatty acid biosynthesis fluxes for the R. jostii RHA1 genomic scale metabolic (GSM) model at various carbon uptake rates for ethanol and acetate.

FIG. 3B is a graphical depiction illustrating flux differences larger than 3 mmol/g DCW/h between ethanol and acetate in a fatty acid biosynthesis simulation. The “rxn” stands for reaction, and the number in each well represents the magnitude of the flux of the corresponding reaction. The negative sign indicates the backward direction of the reaction. OAA: oxaloacetate; PEP: phosphoenolpyruvate; PP: pentose phosphate.

FIG. 3C is a bar graph depicting the dry cell weight (DCW) determined for the indicated samples.

FIG. 3D is a bar graph depicting lipid content in terms of dry cell weight for the indicated samples.

FIG. 3E is a bar graph depicting carbon consumption for the indicated samples.

FIG. 3F is a bar graph depicting cellular ATP level for the indicated samples.

FIG. 3G is a bar graph depicting the pH of culture for the indicated samples.

FIG. 3H is a bar graph depicting the cellular NAD (P) H level for the indicated samples.

FIG. 4 is a categorization and comparison of complex lipid species accumulated in R. jostii RHA1 in the conditions with ethanol or acetate as sole carbon source. 1) comparison between ethanol and acetate conditions with WT strain (WT_ethnoal vs WT_acetate); 2) comparison between WT and AsF strains under ethanol condition (WT_ethanol vs AsF_ethanol); 3) comparison between WT and AsF strains under acetate condition (WT_acetate vs AsF_acetate); 4) comparison between ethanol and acetate conditions with AsF strain (AsF_ethanol vs AsF_acetate). On the left side of each plot, the lipid category is indicated. TAG: triacylglycerols; FA: Fatty Acids and Conjugates; GPE: Glycerophosphoethanolamines; GPI: Glycerophosphoinositols; GroG: Glycerophosphoglycerols; DAG: Diradylglycerols; GroGroG: Glycerophosphoglycerophosphoglycerols; FAcyl: Fatty Acyls; FAE: Fatty esters; GPCho: Glycerophosphocholines; Cer: Ceramides; GSL: Glycosphingolipids; GPL: Glycerophospholipids; GL: Glycerolipids.

FIG. 5A is a schematic depiction of the lipid biosynthetic pathway from ethanol. Genes of ACDH, sthA and FASI were overexpressed with recombinant plasmid method. ACDH, acetaldehyde dehydrogenase; sthA, soluble pyridine nucleotide transhydrogenase; FASI, type I fatty acid synthase.

FIG. 5B is a bar graph depicting the experimental lipid fermentation results using the wildtype (WT) and engineered R. jostii RHA1 strains with ethanol as sole carbon source having the lipid content in dry cell weight.

FIG. 5C is a bar graph depicting carbon consumption for the indicated samples.

FIG. 5D is a bar graph depicting cellular ATP level for the indicated samples.

FIG. 5E is a bar graph depicting cellular NADPH level for the indicated samples.

FIG. 6 is a graph of the pH change of the culture media of the engineered AsF strain when using ethanol, and mixed ethanol and acetate as sole carbon sources. Ethanol means ethanol as used as sole carbon source; Mix_4:1 means a mixed C₂ carbon source with 4:1 mole ratio of ethanol and acetate; Mix_2:1 means a mixed C₂ carbon source with 2:1 mole ratio of ethanol and acetate.

FIG. 7A is a schematic illustration of an integrated electro-bio-fuel system. SSF stands for single stage fermentation mode (SSF) in which the R. jostii RHA1 was inoculated into the system to achieve simultaneous cell growth and lipid accumulation in a one-step process. WWC stands for whole cell catalyst fermentation mode where the RHA1 strains served as the whole cell catalyst for lipid production with limited cell growth. The WCC fermentation was performed for 24 hours with only the first 15 hours being conducted simultaneously with the CO₂ electroreduction.

FIG. 7B depicts graphs of a single stage fermentation (SSF) mode for biomass/lipid production with the wild type R. jostii RHA1 in the integrated system.

FIG. 7C depicts bar graphs of the lipid production of wild type and engineered strain FASI-ACDH3-sthA (AsF) under WCC mode in the integrated system. Cell density was indicated by dry cell weight and lipid production was calculated by deducting the initial lipid accumulation from final lipid titer.

FIG. 7D is a plot of lipid productivity of an electrobio fuel system with the indicated high lipid producing algae and microalgae studies.

### SUMMARY

Disclosed herein is a method for the production of value added chemicals comprising: introducing carbon dioxide to a system comprising an electrochemical cell comprising a carbon dioxide reducing catalyst and an electrolyte under conditions suitable for conversion of carbon dioxide to one or more C₂ compounds; introducing at least a portion of the one or more C₂ compounds to a second system comprising a microbe and a media under conditions suitable for conversion of the C₂ compounds to one or more C₂₊ₙ compounds where n is from about 1 to about 100.

Also disclosed herein is a method of preparing value added chemicals comprising: a means for converting carbon dioxide to one or more C₂ compounds and a microbe wherein the C₂ compound is contacted with the microbe under conditions suitable for the formation of one or more Can compounds.

### DETAILED DESCRIPTION

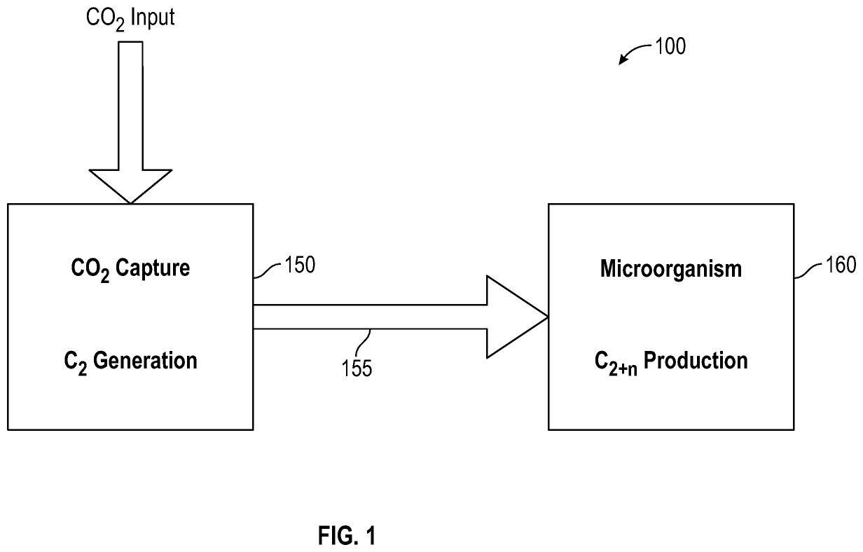

Disclosed herein are compositions, methods and sytems for carbon dioxide capture and conversion of the captured carbon dioxide to a higher value chemical, a higher number of carbon atoms, or precursor thereof. In an aspect, a method of the present disclosure comprises (i) sequestration of carbon dioxide; (ii) conversion of carbon dioxide to a two carbon atom (C₂) compound of the type disclosed herein; and (iii) introduction of the C₂ compound to a microbe able to convert the C₂ compound to a C₂₊ₙ compound where n is greater than 1, alternatively n ranges from about 1 to about 100, alternatively from about 1 to about 50, alternatively from about 1 to about 25 or alternatively from about 1 to about 10. In one or more aspects, a system for enacting the methods of the present disclosure is schematically depicted in FIG. 1.

In one or more aspects, the methods and compositions disclosed herein are used in conjunction with a carbon dioxide capture and conversion system, designated as a CO₂-CCS. With reference to FIG. 1, a CO₂-CCS of the present disclosure 100 comprises a unit 150 in fluid communication with another unit 160 via a coupling mechanism such as a conduit 155 that allows for user directed transfer of at least a portion of the output of unit 150 to unit 160. Unit 160 may produce an output that is subsequently removed from unit 160 and processed to meet one or more user and/or process goals. In one or more aspects, an input to unit 150 is carbon dioxide.

In one or more aspects, disposed within unit 150 is an electrochemical cell and compositions suitable tor the reduction of carbon dioxide to one or more C₂ compounds. Several half reactions for the reduction of carbon dioxide are given in equations 1-6. The one or more C₂ compounds may then serve as the input to unit 160.

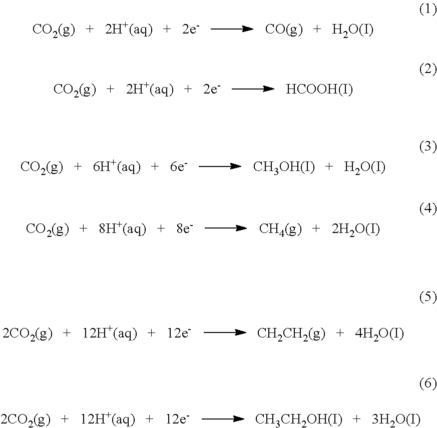

In one or more aspects, disposed within unit 160 are one or more organisms and compositions suitable for conversion of the one or more C₂ compounds to compounds characterized by a carbon atom number of C₂₊ₙ, where n is equal to or greater than about 1, alternatively n ranges from about about 1 to about about 100, alternatively from about 1 to about 50, alternatively from about 1 to about 25 or alternatively from about 1 to about 10, alternatively, 2, 3, 4, 5, 6, 7, 8, 9, 10, 11, 12, 13, 14, 15, 16, 17, 18, 19, 20, or combinations thereof. Each of these units (i.e., 150, 160) along with compositions, apparati and methods assoiated with these units and the CO₂-CCS will be described in more detail later herein.

In an aspect, carbon dioxide for use in the present disclosure may be obtained from any suitable source. Nonlimiting examples of sources of the carbon dioxide include burning fossil fuels; large point sources such as chemical plants; direct air capture; and combinations thereof. In one or more aspects, carbon dioxide input to the CO₂-CCS may originate from one or more materials and/or devices designed to capture CO₂ from any suitable source and subject the captured CO₂ to one or more purification techniques such as scrubbing the gas by passing over a catalyst to remove residual hydrocarbons or other impurities that could affect the compositions and/or other components of the CO₂-CCS. In one or more aspects, the captured CO₂ is pressurized and/or liquified and/or purified prior to being introduced to the CO₂-CCS. Consequently, the CO₂ entering the CO₂-CCS is hereinafter referred to as a purified CO₂.

In one or more aspects, a purified CO₂ is introduced to a unit (FIG. 1 unit 150) where disposed within the unit is an electrochemical cell that is operated under conditions suitable for reduction of carbon dioxide. In one or more aspects, the electrochemical cell comprises a cathode part and an anode part. Without wishing to be limited by theory, at the cathode part the CO₂ molecule is directly reduced to other chemicals (e.g., CO, CH₃OH and C₂H₄ etc.) faciliated by an electrochemical CO₂ reduction reaction catalyst designated CO₂RR. In one or more aspects, a CO₂-CCS is further characterized by a unit having a microbe disposed therein incorporating an asymmetrical double-chamber configuration design for separation of CO₂RR and bioconversion. In such aspects, the unit may be further characterized as being equipped with a first chamber having one or more chambers that can be disposed within a larger unit for maintaining the diffusion force of the C2+ products and a second chamber characterized by having a least one dimension larger than the first chamber where microbial fermentation is carried out in the second chamber.

In one or more aspects, the CO₂RR comprises a transition metal such as Ag, Au, Cu, Zn, or combinations thereof. In some aspects, the CO₂RR further comprises a support material such as carbon, silica or alumina where at least a portion of the transition metal is associated with the surface of the support material. In one or more aspects, the support material is porous. In an aspect, the CO₂RR catalyst comprises a supported copper catalyst, alternatively a supported sputtered copper catalyst, or alternatively a sputtered copper on a porous support (e.g., carbon support), or combinations thereof. In other aspects, the CO₂RR catalyst comprises a nanoporous Cu catalyst, a nanoporous Cu-M catalyst, or a combination thereof where M is a metal selected from the group consisting essentially of Pt, Ir, Pd, Ag, Au, Rh, Ru, Zn, Sn, Ni, Fe, Re, Ga, In, Cd, TI, Ti, and combinations thereof.

With wishing to be limited by theory, within the electrochemical cell, at the anode part a counter oxidation reaction takes place (e.g., water oxidation in aqueous system). In one or more aspects the cathode and anode parts of the electrochemical cell are present together in the same reaction medium. In other aspects, the cathode and anode parts are separated by a membrane. Nonlimiting examples of membranes suitable for use in the electrochemical cell include proton, anion, and bipolar membranes.

In one or more aspects, a purified CO₂ (for e.g., CO₂ in gas form introduced as a stream) is subjected to an electrochemical reduction using an electrochemical cell of the type disclosed herein. Subsequent to electrochemical reduction, the CO₂ reduction products include for example a C₂-C₃ alkane, a C₂-C₃ alkene, a C₂-C₃ alcohol or combinations thereof. Nonlimiting examples of CO₂ reduction products include carbon monoxide (CO), formic acid (HCOOH), methanol (CH₃OH), formaldehyde (HCHO), methane (CH₄), ethylene (C₂H₄), ethanol (CH₃CH₂OH), acetate (C₂H₂O₂⁻), formaldehyde (HCHO), methane (CH₄), ethylene (C₂H₄) and combinations thereof. In one or more aspects, the electrochemical reduction is carried out under conditions that favor the formation of ethanol (CH₃CH₂OH), acetate (C₂H₂O₂⁻) or combinations thereof.

In one or more aspects, at least a portion of the C₂ product from CO₂ reduction (e.g., ethanol, acetate) is conveyed to a unit (FIG. 1, unit 160) having disposed therein a suitable media and microbe capable of converting the C₂ product to a C₂ₙ₊₁ product. In an aspect, the method of transferring the C₂ product comprises unidirectional passage of the C₂ product from unit 150 to unit 160.

In an aspect, a suitable media comprises one or more of the electrolyte solutions of the reactor from which the C₂ product has been transferred. In one or more aspects, any microbe capable of converting the C₂ product to a C₂ₙ₊₁ product may be employed. A microbe suitable for use may be characterized as a wild-type or an engineered microbe that uses acetyl-CoA as the central metabolite. In an aspect, the microbe is a bacteria, fungus, or yeast. In one or more aspects, the microbe is a wild type organism. Alternatively, the microbe has been genetically engineered to more effectively convert the C₂ product to one or more C₂ₙ₊₁ products. Genetic engineering of the microbe to meet one or more process and/or user goals may be carried out using any suitable technology know to one of ordinary skill in the art for the mutation of microbes. Nonlimiting examples of microbes suitable for use in the present disclosure include the microbes Tetraselmis sp., Chaetoceros sp., Pseudomonas putida, Rhodococcus jostii or combinations thereof.

In an aspect, a method of the present disclosure comprises fermenting the mixture of C₂ products with a microbe under conditions suitable for the formation one or more C₂₊ₙ products. In an aspect, the C₂₊ₙ product is selected from the group consisting of polymers, fuels, lipids, light olefins, gluconic acid, lactic acid, malonic acid, propionic acid, triacids, citric acid, aconitic acid, xylonic acid, acetoin, furfural, levoglucosan, lysine, serine, threonine, 1,4 succinic, fumaric acid, malic acid, 2,5 furan dicarboxylic acid, 3-hydroxy propionic acid, aspartic acid, glucaric acid, glutamic acid, itaconic acid levulinic acid, 3-hydroxybutyrolactone, glycerol, sorbitol, xylitol, arabinitol, 5-hydroxymethylfurfural, ferulic acid, terpenoids, polyketides, carotenoids, polyphenols, alkaloids and combinations thereof.

In one or more aspects, an exemplary CO₂-CCS comprises an electro-microbial conversion with C₂ (EMC2). An efficient EMC2 system may comprise a four tier design to achieve integration of the electrochemical CO₂ reduction reaction (CO₂RR) and microbial conversion of the C₂ product. The first tier may focus on electrocatalysis, where the design and selection of electrolyzer (electrochemical cell), catalysts, and electrolytes ensure the efficient electrocatalysis, facilitated by CO2RR, to C₂ products under biocompatible conditions. The electrochemical cell, catalysts, and electrolytes may be of the type disclosed herein.

The second tier is the design of a chem-bio interface to make sure that microbial conversion does not interfere with CO2RR. The third tier is the design of an integrated EMC2 system to achieve efficient mass transfer of C₂ products between electrocatalysis and fermentation. The fourth tier is a microbial design to efficiently channel the C₂ compounds to the tricarboxylic acid (TCA) cycle of the microbe and to enhance the carbon flux toward target bioproducts. The four-tier design may achieve complete and efficient electrotrophic microbial conversion of CO₂. As the starting point to convert CO₂ to C₂ intermediates, the electrocatalysts disposed within the electrochemical cell may be designed to provide sufficient C₂ products for microbial conversion under bioamicable conditions.

These criteria are minimally met with the integrated design and selection of electrolyzer, electrolytes, and catalysts. First, a flow electrolyzer equipped with gas diffusion electrodes (GDEs) may be used for C₂ production, considering the potentially high yield of C₂ products. Without wishing to be limited by theory, a GDE setting can achieve much higher current density and C₂ product content than the conventional electrocatalysis with H-cell, as the CO₂ feeding to GDE is not limited.

In another exemplary aspect, the ECM2 operates when CO₂ is introduced to a first reactor which comprises a flow electrolyzer equipped with GDEs. The first reactor may further comprise an electrolyte (e.g., salt solution) characterized by its ability to flow between the first electrocatalysis reactor a and second reactor in which one or more bioconversion reactions occur. In one or more aspects, the first reactor and second reactor are in fluid communication with each other via a unidirectional membrane that affords movement from the first reactor to the second reactor while flow from the second reactor to the first reactor is restricted. The electrolyte used in this system may be suited for both electrocatalysis and cell cultivation. In an aspect, the electrolyte is a “basal” solution comprising phosphate buffer (e.g., NaH₂PO₄+K₂HPO₄+NaCl) with a pH of about 7.

In an aspect, disposed within the first reactor is one or more CO₂RR catalysts which facilitates the production of a C₂ product such as ethanol, acetate or a combination thereof. The C2 product may be conveyed to a second reactor comprising the salt solution of the first reactor and one or more microbes capable of conversion of the C₂ product to a C₂₊ₙ compound. In an aspect, the microbe is a wildtype species of said organism. In the alternative, the microbe is genetically modified to facilitate the production of the C₂₊ₙ compound from the C₂ product in order to meet one or more user and/or process goals. A nonlimiting example of a microbe suitable for use in the present disclosure is Pseudomonas putida. In an aspect, a method of the present disclosure comprises fermenting the mixture of C₂ products with a microbe under conditions suitable for the formation one or more C₂₊ₙ products.

The methods and processes disclosed herein overcome the barriers historically associated with the systematic design of electrocatalysis, chemical-biological interface, and microbes to enable efficient EMC2 intermediates. The soluble C₂ intermediates disclosed herein can facilitate rapid mass transfer, readily enter primary metabolism, have less toxicity, carry more energy and electrons, and serve as better molecular building blocks for many microbes. The multitier chem-Bio interface design allowed the EMC2 system to achieve from about 2 fold to about 10 fold increase of microbial biomass productivity compared to C₁ intermediate and hydrogen-driven routes, respectively; alternatively, from about 2 to about 5 fold increase or alternatively about a 5 fold increase.

In one aspect, the presently disclosed methods function as a multimodule synthetic biology design which can produce medium-chain-length PHA (polyhydroxyalkanoates), biodegradable polymers, representing much higher productivity and molecular chain length than platforms based on C₁ intermediates, hydrogen, or electrons.

### ADDITIONAL DISCLOSURE

The following enumerated aspects of the present disclosures are provided as non-limiting examples.

A first aspect which is a method for the production of value added chemicals comprising: introducing carbon dioxide to a system comprising an electrochemical cell comprising a carbon dioxide reducing catalyst and an electrolyte under conditions suitable for conversion of carbon dioxide to one or more C₂ compounds; introducing at least a portion of the one or more C₂ compounds to a second system comprising a microbe and a media under conditions suitable for conversion of the C₂ compounds to one or more C₂₊ₙ compounds where n is from about 1 to about 100.

A second aspect which is the method of the first aspect wherein the one or more C₂ compounds comprise ethylene (C₂H₄), ethanol (CH₃CH₂OH), acetate (C₂H₂O₂⁻), ethylene (C₂H₄) or combinations thereof.

A third aspect which is the method of any of the first through second aspects wherein the C₂ compound comprises ethanol, acetate or combinations thereof.

A fourth aspect which is the method of any of the first through third aspects wherein the catalyst comprises Ag, Au, Cu, Zn or combinations thereof.

A fifth aspect which is the method of any of the first through fourth aspects wherein the catalyst comprises a support material.

A sixth aspect which is the method of any of the first through fifth aspects wherein the support material is porous.

A seventh aspect which is the method of any of the first through sixth aspects wherein the catalyst comprises a supported copper catalyst.

An eighth aspect which is the method of any of the first through seventh aspects wherein the catalyst comprises a nanoporous Cu-M catalyst M is a metal selected from the group consisting ofPt, Ir, Pd, Ag, Au, Rh, Ru, Zn, Sn, Ni, Fe, Re, Ga, In, Cd, TI, Ti, and combinations thereof.

A ninth aspect which is the method of any of the first through eighth aspects wherein the media and electrolyte are compositionally the same.

A tenth aspect which is the method of any of the first through ninth aspects wherein the microbe is a wildtype strain.

An eleventh aspect which is the method of any of the first through ninth aspects wherein the microbe is a mutant strain.

A twelfth aspect which is the method of any of the first through eleventh aspects wherein the one or more C₂₊ₙ compounds is selected from the group consisting of polymers, fuels, lipids, light olefins, gluconic acid, lactic acid, malonic acid, propionic acid, triacids, citric acid, aconitic acid, xylonic acid, acetoin, furfural, levoglucosan, lysine, serine, threonine, 1,4 succinic, fumaric acid, malic acid, 2,5 furan dicarboxylic acid, 3-hydroxy propionic acid, aspartic acid, glucaric acid, glutamic acid, itaconic acid levulinic acid, 3-hydroxybutyrolactone, glycerol, sorbitol, xylitol, arabinitol, 5-hydroxymethylfurfural, ferulic acid, terpenoids, polyketides, carotenoids, polyphenols, alkaloids and combinations thereof.

A thirteenth aspect which is the method of any of the first through twelfth aspects wherein the one or more C₂₊ₙ compounds comprise lipids.

A fourteenth aspect which is a method of preparing value added chemicals comprising: a means for converting carbon dioxide to one or more C₂ compounds and a microbe wherein the C₂ compound is contacted with the microbe under conditions suitable for the formation of one or more C₂ compounds.

A fifteenth aspect which is the method of the fourteenth aspect wherein the means for converting carbon dioxide to one or more C₂ compounds is an electrochemical cell comprising a nanoporous Cu-M catalyst where M is a metal selected from the group comprising Pt, Ir, Pd, Ag, Au, Rh, Ru, Zn, Sn, Ni, Fe, Re, Ga, In, Cd, TI, Ti and combinations thereof.

A sixteenth aspect which is the method of any of the fourteenth through fifteenth aspects wherein the C₂ compound comprises ethanol, acetate or combinations thereof.

A seventeenth aspect which is the method of any of the fourteenth through sixteenth aspects wherein the microbe comprises a bacteria.

An eighteenth aspect which is the method of any of the fourteenth through seventeenth aspects wherein the microbe is mutated.

A nineteenth aspect which is the method of any of the fourteenth through eighteenth aspects+wherein the one or more C₂₊ₙ compounds is selected from the group consisting of polymers, fuels, lipids, light olefins, gluconic acid, lactic acid, malonic acid, propionic acid, triacids, citric acid, aconitic acid, xylonic acid, acetoin, furfural, levoglucosan, lysine, serine, threonine, 1,4 succinic, fumaric acid, malic acid, 2,5 furan dicarboxylic acid, 3-hydroxy propionic acid, aspartic acid, glucaric acid, glutamic acid, itaconic acid levulinic acid, 3-hydroxybutyrolactone, glycerol, sorbitol, xylitol, arabinitol, 5-hydroxymethylfurfural, ferulic acid, terpenoids, polyketides, carotenoids, polyphenols, alkaloids and combinations thereof.

A twentieth aspect which is the method of any of the fourteenth through nineteenth aspects wherein the one or more C₂₊ₙ compounds comprise lipids.

### EXAMPLES

In the following examples, RLU means relative luminescence units and RAU means relative absorbance units. All the data was collected with biological triplicates. All the values are presented in the form of mean±SEM (standard error of the mean).

### Example 1

### C₂ Compounds as Precursors for Lipid Biosynthesis

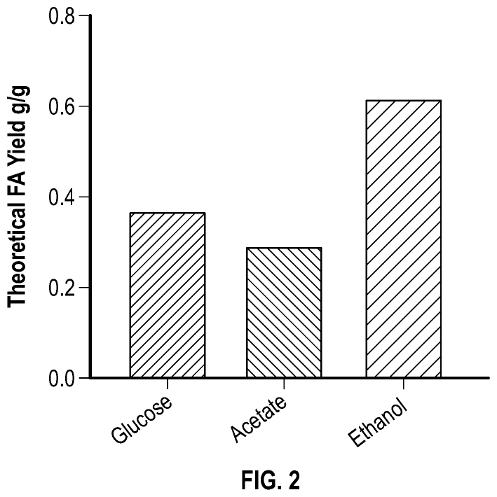

The potential limitations of oleaginous organisms for the production of electrobiofuel (EBF) were investigated. In these experiments, ethanol and acetate were used as the C₂ carbon source. Fatty acids (FA) are the precursor of lipids, going through the Kennedy pathway to serve as the building blocks for the synthesis of triacylglycerol, which is the primary component in lipids used for biodiesel production. A theoretical calculation showed that acetate has a theoretical FA yield of 0.29 g FA/g acetate, comparable to glucose's 37 g FA/g glucose, based on the energy content of the substrates. Ethanol's theoretical fatty acid yield is even higher, reaching 0.62 g FAs/g ethanol, based on the same calculation, FIG. 2.

This comparison suggests a great energy potential of the C₂ compounds, particularly ethanol, as a feedstock for microbial fuel production. Considering that FA biosynthesis is influenced not only by substrate energy content but also by the microbe specificity and metabolic processes the impact of C₂ metabolism on FA biosynthesis was further investigated.

### Example 2

A genome-scale metabolic (GSM) model that is widely used to describe metabolism under different conditions and simulate the metabolic fluxes of target bioproducts were used. To study the C₂ metabolism on FA biosynthesis, a draft GSM model was constructed using the KBase platform for the model oleaginous bacteria R. jostii RHA1 to grow in customized minimum media with either ethanol or acetate as the sole carbon source, The draft metabolic model consisted of 1284 compounds and a total of 1328 reactions, with 56 reactions added through the default gapfilling approach to ensure the biomass production in the specified media. Then, the flux balance analysis (FBA) with FA biosynthesis as objective function was performed under a series of carbon uptake rates to simulate FA biosynthesis under different carbon inputs. The simulation results indicated that, as carbon input increased there is corresponding increase in fatty acid biosynthesis for both ethanol and acetate, see FIG. 2.

Additionally, in the region of carbon uptake rate from 15 to 50 mmol/g dry cell weight (DCW)/h ethanol can support a higher rate of fatty acid synthesis compared to acetate, aligning with the theoretical predictions of higher fatty acid yields from ethanol than from acetate.

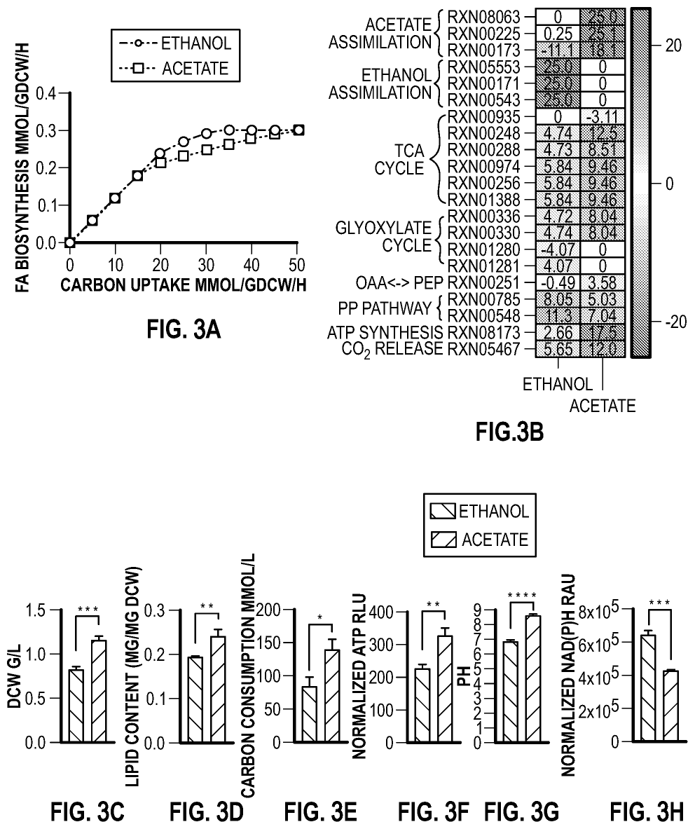

To identify the metabolic pathways contributing to the difference in FA biosynthesis between the two carbon sources, the flux of all reactions in their respective FBA results were compared. Reactions with a flux difference greater than 3 mmol/gDCW/h were identified as this threshold was considered to be significant relative to most reactions with fluxes below 10 mmol/gDCW/h. The investigations showed when using ethanol, acetyl-CoA is supplied via ethanol oxidation, whereas with acetate as the carbon input, acetyl-CoA is produced through acetate phosphorylation, FIG. 3. The notable difference between these two carbon assimilation pathways is that the former generates a substantial amount of reducing equivalents (e.g. NADH), which can be utilized for various biosynthesis processes including fatty acid (FA) synthesis. In contrast, the latter does not produce any reducing equivalents but consumes one ATP. Consistently, under the acetate condition, a higher flux of acetyl-CoA entered the tricarboxylic acid (TCA) cycle to support the generation of reducing equivalents and ATP, compared to the ethanol condition. Additionally, the ATP generation conducted by ATPase was also in a higher level under the acetate condition than ethanol condition. These higher levels of energy metabolism, including the TCA cycle and oxidative phosphorylation, can explain the higher CO₂ release and can lead to a diversion of carbon flux away from FA biosynthesis. Moreover, acetate has also led to higher carbon flux in the glyoxylate shunt and the phosphoenolpyruvate/pyruvate/oxaloacetate node, which are known for their capability to utilize acetyl-CoA from FA oxidation for gluconeogenesis. These carbon flux distributions could have collectively contributed to the lower flux of acetyl-CoA entering the initial reactions of FA biosynthesis, where acetyl-CoA is converted into malonyl-CoA and then malonyl-ACP.

In contrast to acetate, the flux distribution with ethanol as carbon input showed a higher flux in pentose phosphate (PP) pathway which is known supply NADPH in Rhodococcus species. Additionally, under the ethanol condition, a reversible reaction in glyoxylate cycle was upregulated in both the forward and backward directions, leading to NADH consumption and NADPH generation. Since FA biosynthesis requires NADPH rather than NADH, these reactions could have contributed to the higher FA biosynthesis from ethanol by providing the necessary NADPH. On the other hand, the simulation showed the optimal FA biosynthesis from ethanol occurs through the direct conversion of acetaldehyde to acetyl-CoA, bypassing the pathway of acetaldehyde-acetate-acetylphosphate-acetyl-CoA, which requires one additional ATP consumption. Interestingly, under the ethanol condition, there is a significant flux from acetyl-CoA to acetylphosphate, indicating an excess of acetyl-CoA derived from ethanol. These results suggested that enhancing the pathway of ethanol-acetaldehyde-acetyl-CoA-FA could be a factor in FA biosynthesis from ethanol. Overall, the metabolic simulation indicated that ethanol has the potential to support higher acetyl-CoA flux, provide more reducing power for FA biosynthesis, and reduce carbon loss compared to acetate, due to its higher energy content. To experimentally verify the potential of ethanol as a carbon source for fatty acid biosynthesis and lipid production, a lipid fermentation experiment was conducted on the model bacteria R. jostii RHA1 using ethanol or acetate as the sole carbon source. Surprisingly, with the same total carbon supply of 225 mmol/L, the acetate can support 1.162±0.043 g DCW/L of the RHA1 dry cell weight while the ethanol can only support 0.830±0.031 g DCW/L, FIG. 3C-H. Moreover, the lipid contents quantified in the form of fatty acid methyl esters showed 0.241±0.013 g lipid/g DCW under the acetate condition whilst only 0.194±0.002 g lipid/g DCW under the ethanol condition. This observation contrasts with the results of theoretical calculation and metabolic simulation, indicating the presence of metabolic factors that hindered the conversion of ethanol into lipids.

To identify the limiting factors, the levels of carbon influx, ATP, and NAD(P)H was analyzed as these are factors known to influence lipid biosynthesis. Firstly, the RHA1 strain had a significantly lower carbon consumption rate with ethanol at 84.4±14.7 mmol/L compared to acetate at 139.7±15.8 mmol/L. The lower ethanol uptake could limit the carbon flux entering FA and lipid biosynthesis. Secondly, the cellular ATP level in R. jostii RHA1 was also significantly lower in the presence of ethanol as sole carbon source compared to acetate. The lower carbon intake of ethanol could have limited the ATP production level. Fermentation with ethanol resulted in the appearance of 8.27±0.71 mmol/L acetic acid in the culture media and a significantly lower pH compared to the fermentation with acetate. This observation suggested an acetic acid accumulation in the R. jostii RHA1 during ethanol metabolism, which can cause internal cytoplasmic acidification and further affect ATP synthase activity on the cytoplasmic membrane. R. jostii RHA1 can efflux protons to extracellular space at the expense of ATP in response to the intracellular acidification. This typical microbial response to acid stress can explain the phenomena of pH decrease from 7.5±0.0 to 6.8±0.1 during fermentation with ethanol and contribute to the lower cellular ATP level as well. Thirdly, the NAD (P) H level in the R. jostii RHA1 under ethanol condition is about 2.2-folds higher than that in acetate conditionsupporting the ethanol assimilation reactions in the metabolic simulation, which produces abundant reducing equivalents. Of particular interest is the imbalance between the high NAD (P) H level and low ATP level when using ethanol as carbon source suggesting an inefficient transformation of NAD (P) H into ATP. This phenomenon implies a “saturation” of the respiratory chain, potentially due to the cytoplasmic acidification that have impacted the efficiency of the electron transport. Overall, compared with acetate, ethanol supported higher reducing power generation in R. jostii RHA1, but the ethanol metabolism caused cellular acidification stress that significantly affected the carbon intake and ATP production, which impacted the lipid production.

### Example 3

The FBA simulation demonstrated the advantages of ethanol over acetate for FA biosynthesis, but the observed differences in lipid accumulation compared to the simulation suggest that the actual metabolic flux distribution when using these two C2 substrates may deviate from the simulated scenario. Therefore, a comprehensive metabolomics study with measurements of both primary metabolites and complex lipid species was conducted to investigate the actual metabolic distributions in R. jostii RHA1 when using ethanol or acetate as sole carbon source. On one hand, among a total of 127 identified primary metabolites in R. jostii RH1, 79 metabolites were found that showed significantly different levels (Abslog2(FC)>1) between the ethanol and acetate conditions. 88.6% of these 79 metabolites exhibited higher levels in the ethanol condition compared to acetate, with most belonged to categories such as amino acids, monosaccharides, disaccharides, and fatty acids. To visualize the pathways involved, all these differential metabolites were mapped onto the metabolic pathways of R. jostii RHA1 using the cellular overview tool in the BioCyc database. It showed that these differential metabolites mainly involved in glycolysis, pentose phosphate pathway, trehalose biosynthesis, and nucleoside and amino acid biosynthesis.

The significantly different metabolic flux distribution of primary metabolism between ethanol and acetate condition could be attributed to the acidification phenomenon observed in ethanol metabolism. First, under the ethanol condition, a higher level of trehalose was observed whose accumulation is known to protect microbes from acidification stress. Second, under the ethanol condition, a higher level of nicotinamide was observed, indicating an upregulation of the salvage pathway of nicotinamide adenine dinucleotide (NAD) In addition, the aspartate, which is the precursor for de novo NAD biosynthesis, also exhibited a significantly higher level under the ethanol condition. The two observations indicated an increased requirement for NAD when ethanol is utilized as the primary carbon source. It can be explained by the metabolic requirement for NAD when metabolizing aldehydes and alcohols. Moreover, the upregulation of the NAD salvage pathway can increase cellular tolerance to aldehydes and acetic acid. Thirdly, a broad upregulation in the level of diverse amino acids was observed under the ethanol condition compared to the acetate condition. On one hand, the higher level of amino acids can enter tRNA charging reactions to boost the protein synthesis which is important for acid tolerance. On the other hand, these amino acids can undergo decarboxylation which consume intracellular protons to increase the acid tolerance of microbial cells. Overall, these results demonstrated the different primary metabolism responses of the RHA1 between ethanol and acetate carbon sources, highlighting a significantly upregulated carbon flux towards primary metabolic pathways under the ethanol condition compared to the acetate condition.

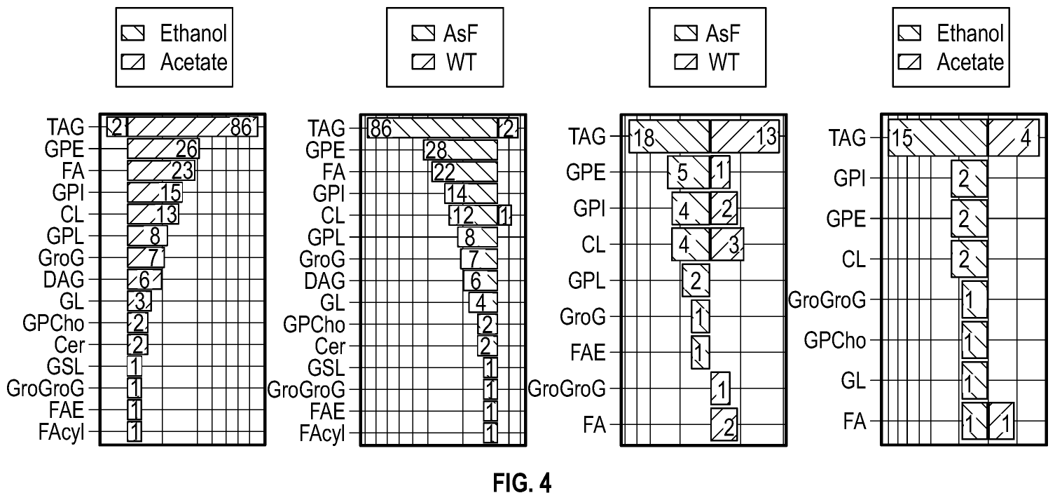

One the other hand, the findings from lipidomic experiment revealed among the total 253 identified lipid compounds, 197 exhibited significantly higher levels in the acetate condition, whereas only 2 were higher in the ethanol condition (t-test, p<0.05). This result indicated a significant and widespread reduction in lipid synthesis when ethanol is used as the carbon source compared to the condition with acetate. Specifically, among these 197 compounds, 86 were triradylglycerols (TAG), 23 were fatty acids (FA), 3 were glycerolipids (GL), and 1 was a fatty ester (FAE), FIG. 4. These lipid types are essential for energy storage in microbial organisms and serve as the primary components for biofuel production. Additionally, the levels of phospholipids including glycerophosphoethanolamines (GPE), glycerophosphoinositols (GPI), glycerophosphoglycerols (GroG), diradylglycerols (DAG), and glycerophospholipids (GPL), were also dramatically decreased, which could potentially disrupt the biogenesis and structural integrity of cellular membranes. This result indicated the homeostasis of lipid metabolism were perturbed when ethanol served as sole carbon source, further supporting to the acidification effect of ethanol. By integrating the findings from lipidomic analysis and primary metabolism, it was evident that compared to the acetate, ethanol supported a higher metabolic flux in diverse primary metabolic pathways to produce energy and building blocks which however entered synthetic pathways such as amino acid and nucleoside biosynthesis rather than lipid biosynthesis.

### Example 4

Metabolic Manipulation to Enhance Lipid Production from C₂ Compounds

A comparative analysis between metabolic simulation and metabolomic results was used in order to improve lipid conversion from C₂ substrates by genetic engineering. Firstly, the simulation did not consider the metabolic impact of acidification stress during ethanol metabolism which has significantly increased the biosynthesis of amino acids, nucleosides, and trehalose therefore can deviate carbon flux from FA and lipid biosynthesis. Secondly, the simulation suggested an ethanol assimilation pathway of ethanol-acetaldehyde-acetyl-CoA which does not require ATP consumption. However, in the actual scenario there is notable acetic acid accumulation when ethanol serves as sole carbon source, indicating ethanol could have been quickly oxidized into acetic acid and which then be converted into acetyl-CoA by acetyl-CoA synthetases at expenses of two units of ATP. Thirdly, ethanol supported significantly higher reducing power than acetate. In the simulation, although a portion of NADH was transformed into NADPH to supply NADPH for FA biosynthesis, there are still large amount of NADH were dissipated by ferric ion reduction and export, indicating an insufficient utilization of the energy from ethanol to FA. Based on this comparative analysis, a genetic engineering approach to redirect carbon flux from other biosynthetic pathways to fatty acid biosynthesis, reduce acetic acid accumulation from ethanol oxidation, improve ATP supply, and improve reducing power utilization for lipid biosynthesis was explored.

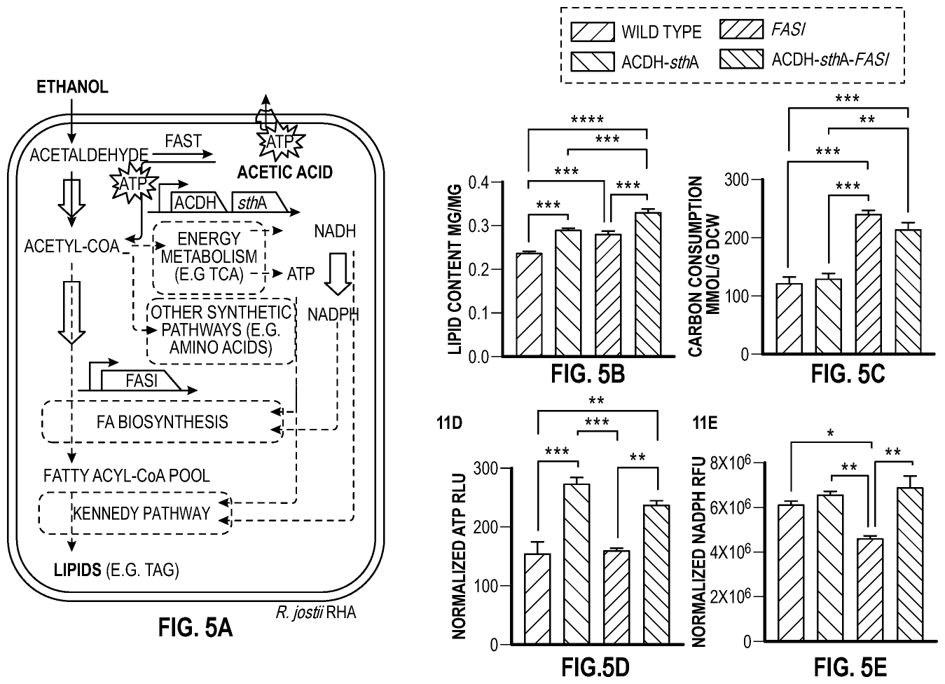

First, to reduce ATP cost and acetic acid accumulation during ehanol assimilation, the gene ACDH3 encoding the acetaldehyde dehydrogenase was overexpressed to convert acetaldehyde to acetyl-CoA and circumvent the generation of acetate, FIG. 5. The upregulation in this reaction can compete with the rapid acetaldehyde-to-acetic acid oxidation, enabling the acetyl-CoA generation without extra ATP consumption and mitigation of acetic acid accumulation. Second, given that R. jostii RHA1 employs NADPH instead of NADH for fatty acid biosynthesis, sthA, a soluble pyridine nucleotide transhydrogenase, which converts NADH into NADPH, was simultaneously overexpressed to channel reducing power into fatty acid biosynthesis. Co-overexpression of the ACDH and sthA significantly improved the intracellular levels of ATP by 78.4%±25.8%. However, the NADPH level is not significantly improved, probably due to the relatively high NADPH level when using ethanol as carbon source. Moreover, this co-overexpression increased the lipid content by 21.6%±0.7%, compared to the wild type (WT) R. jostii RHA1 strain/Meanwhile, the ACDH-sthA strain exhibited a comparable ethanol consumption to that of the WT strain, indicating the ACDH-sthA overexpression has improved the carbon conversion efficiency to lipid. Third, the FASI gene that encodes type I fatty acid synthase (FASI) was overexpressed to channel more carbon flux into FA biosynthesis by taking advantage of the previous observation that the FASI was significantly upregulated when Rhodococcus species accumulated lipids. FASI is a single large, multiunit, and multifunctional enzyme complex that can conduct the condensation and elongation of fatty acids with high efficiency. The FASI strain demonstrated 45.1%±18.7% increase of ethanol uptake and 18.0%±4.1% increase of lipid accumulation, compared to the WT strain. These results indicated that the FAI overexpression not only improved lipid biosynthesis but also increased the capability of the R. jostii RHA1 to assimilate ethanol. Moreover, the FASI overexpression strain exhibited significantly lower levels in both ATP and NADPH compared to the wild-type strain, which could be attributed to the intense consumption of the two factors in lipid biosynthesis. Based on these observations, ACDH and sthA together with FASI was overexpressed in WT strain to support the ATP and NADPH to further enhance lipid production. The levels of ATP and NADPH in the ACDH-sthA-FASI (AsF) strain were both significantly improved by 47.6%±3.5% and 48.0%±20%, respectively when compared with the FAS/strain. At the same time, the ethanol uptake rate was also significantly higher than that in ACDH-sthA strain by 27.5%±14.8%. Moreover, the ACDH-sthA-FASI strain demonstrated 33.1±1.0% of lipid accumulation in total biomass, which was 39.4%±1.5% lipid increase compared to the WT strain. Overall, these results demonstrated that co-overexpression of ACDH, sthA, and FASI genes in R. jostii RHA1 significantly improved its carbon and energy utilization efficiency of ethanol for lipid biosynthesis.

### Example 5

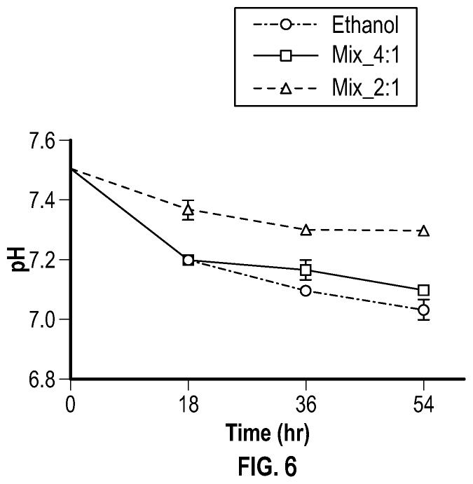

To investigate if the overexpression of ACDH-sthA-FASI redirect the metabolic flux from other synthetic biosynthesis pathway towards lipid biosynthesis, the AsF strain was compared with the WT strain under the ethanol and acetate conditions, for their primary metabolite and complex lipid specie levels. The primary metabolism comparison showed that almost all the primary metabolites involved in carbohydrate metabolism, amino acid and nucleoside biosynthesis were downregulated in the ACDH-sthA-FASI strain under both the ethanol and acetate conditions, indicating a significant lower metabolic flux distribution on these metabolisms. Notably, the high levels of trehalose and amino acids that are important for the WT cells to combat acidification stress when utilizing ethanol were significantly decreased by the ACDH-sthA-FASI overexpression, suggesting that the acidification stress caused by ethanol utilization were mitigated in the AsF strain. This mitigation effect of the ACDH-sthA-FASI overexpression was further evidenced by the less pH decrease and acetic acid accumulation in the culture media of AsF strain compared to WT strain. Overall, it was suggested that the overexpression of ACDH-sthA-FASI significantly mitigated acidification stress and cut down the carbon distribution on energy metabolisms and other synthetic pathways, FIG. 6.

Concurrently, the levels of complex lipid species were also compared between the AsF and WT strains to investigate if ACDH-sthA-FASI overexpression has channeled the metabolic flux into lipid biosynthesis. First, 195 out of the total 253 identified lipid compounds were found significantly higher in the AsF strain than in WT strain under ethanol condition. Notably, these 195 lipid compounds overlap with 99.0% of the 197 lipid compounds that showed significantly lower levels in the WT strain under ethanol condition compared to acetate condition. This indicated that the overexpression of ACDH-sthA-FASI had a remarkable impact on lipid biosynthesis, accurately redirecting the metabolic flux towards increased lipid production. Specifically, 53.8% of these increased lipids belonged to the category of energy storage lipids, including TAG, FA, GL, and FAE. Additionally, phospholipid species such as GPE, GPI, GPL, GroG, and DAG, accounting for 32.3%, were also restored by the ACDH-sthA-FASI overexpression. These increased lipid pools might have played a role in helping RHA1 combat the stress induced during the ethanol assimilation. Second, in the acetate condition, the AsF strain exhibited significantly higher levels of 35 lipid compounds compared to the WT strain, whereas the WT strain only had 20 lipid compounds with higher levels. This indicated that the overexpression of ACDH-sthA-FASI also enhanced the lipid biosynthesis performance of RHA1 when acetate is used as the sole carbon source. Third, when comparing the lipid species of the AsF strain between the ethanol and acetate conditions, it was observed that 25 lipid compounds exhibited significantly higher levels in the ethanol condition, while only 5 lipids showed higher levels in the acetate condition. This finding indicated that ethanol is more efficient in supporting lipid biosynthesis in the AsF strain, further highlighting the effect of ACDH-sthA-FASI overexpression on improving the capability of RHA1 to utilize ethanol for lipid production. Overall, the overexpression of ACDH, sthA, and FASI in RHA1 strain effectively addressed the acidification issue associated with ethanol utilization, significantly improved carbon and energy utilization from both ethanol and acetate sources, and remarkably enhanced lipid production by channeling higher portions of carbon and reducing power into lipid biosynthesis.

### Example 6

### Co-Substrate Effect on Lipid Production Guides Catalyst Design to Tune C2 Profile

Even though, genetic engineering had improved the capability of R.jostii RHA1 to utilize ethanol as carbon source to produce lipid, as depicted in the FIG. 4, the engineered strain AsF still experienced acidification stress and accumulation of acetic acid during ethanol metabolism. Considering the observed alkalizing effect on the culture media, where the pH increased from 7.5±0.0 to 8.7±0.1 when acetate was used as sole carbon source it was speculated that the acetate coproduced with the ethanol from the CO2RR can help to mitigate the acidification stress and further improve lipid production. The potential co-substrate effect of ethanol and acetate for bioconversion, which could be an important factor a electro-microbial conversion system using C₂ as the intermediates was investigated. Specifically, lipid fermentation experiments using the engineered AsF strain with three different C₂ substrate pools: sole ethanol, ethanol: acetate in a ratio of 4:1 (mole: mole), and ethanol: acetate in a ratio of 2:1 (mole: mole), while maintaining the total mole concentration constant were carried out. The results showed that the combination of mixed C₂ (ethanol and acetate) significantly enhanced lipid production compared to using ethanol alone as the carbon source. Under a 4:1 mole ratio of ethanol to acetate, the lipid titer reached 317.9±20.8 mg/L, showing a remarkable 29.5% increase compared to the sole ethanol condition. When the acetate ratio increased to one third of the total carbon source, the lipid titer further improved to 392.7±34.7 mg/L, which is about 60.0% enhancement over the sole ethanol condition. Notably, the AsF strain consumed a similar total carbon source under the three carbon source conditions, indicating that the mixed C₂ of ethanol and acetate led to better carbon and energy utilization for lipid production. This improved efficiency could be attributed to reduced acidification stress when acetate was co-metabolized with ethanol, which resulted in less pH decreases in the culture media during cell growth and lipid accumulation. Overall, these results evidenced that coproducing acetate with ethanol from the CO2RR can improve the lipid production performance of the RHA1 strain and provide guidance for the following catalyst design to tune C₂ production profile.

### Example 7

Integrated System to Enable Rapid Lipid Production from CO₂

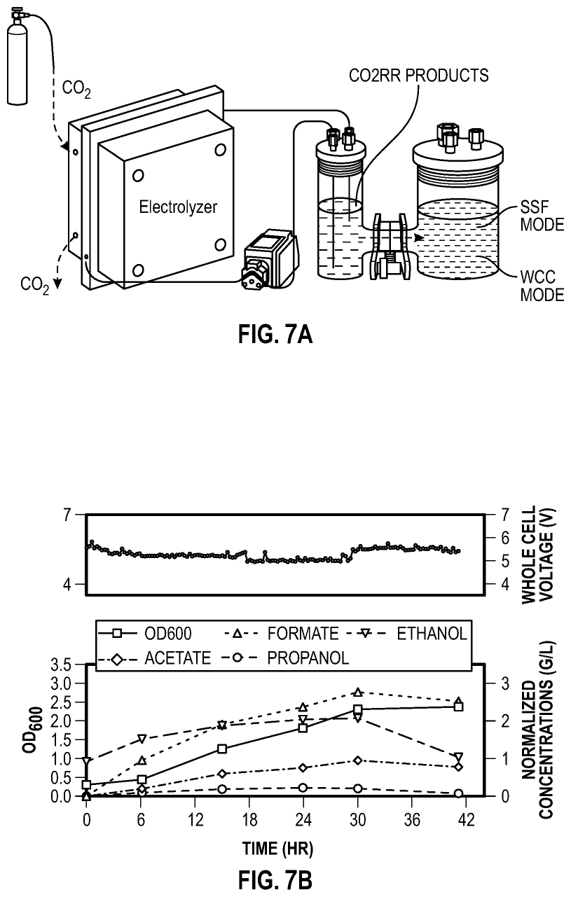

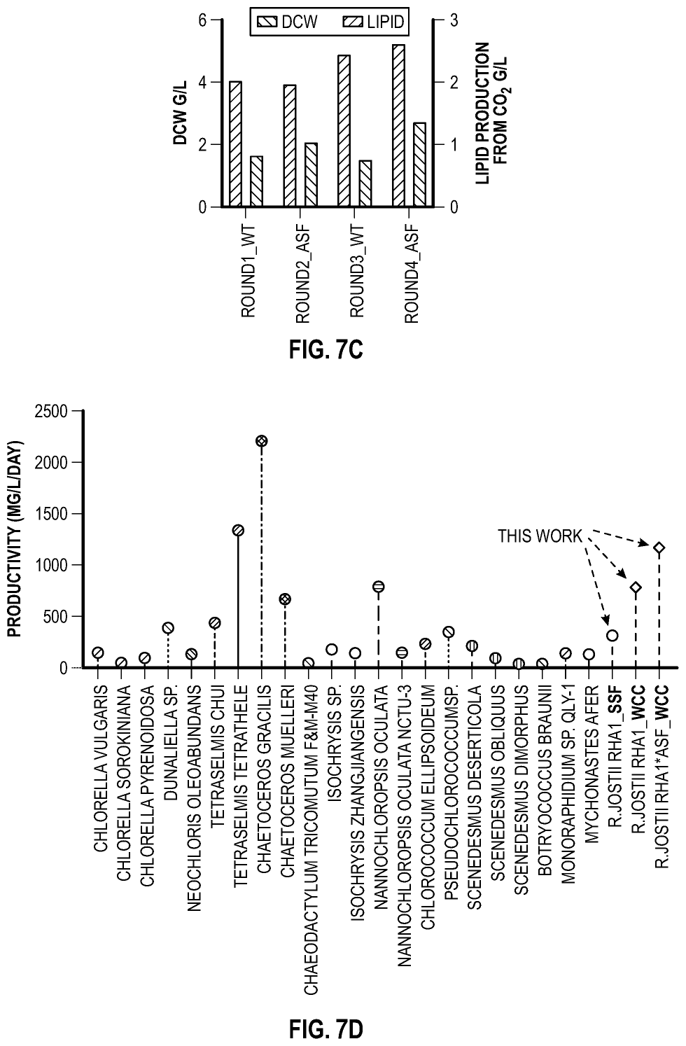

An EBF system was prepared comprising a CO₂ reduction metal catalyst and microbial cell factories in order to investigate lipid production directly from CO₂. The system enabled the entry of finely tuned C₂ products from the CO₂ electrolyzer into the circulation chamber of the bioreactor, where they diffused to the fermentation chamber to support lipid production by the RHA1 cell factories, FIG. 7. To evaluate the performance of the EBF system for CO₂ conversion and lipid production, two commonly used fermentation modes were adopted: single stage fermentation (SSF) mode and whole cell catalyst (WCC) mode to conduct lipid fermentation with RHA1 strains in integrated system. First, in SSF mode, R.jostii RHA1 were inoculated in the integrated system for simultaneous cell growth and lipid accumulation in one process. This fermentation mode was designed to achieve both biomass and lipid production from CO₂ in one step, making it a suitable approach for evaluating the performance and potential of the EBF system for CO₂ conversion and biological system support. The results showed that the EBF system could conduct the SSF fermentation for 42 hours while maintaining stable C₂ concentration and rapid cell growth. Specifically, the voltage of the system remained constant between 5 and 6 volts across the time, and the concentrations of ethanol and acetate kept increasing in the first 30 hours, indicating the metal catalyst was producing sufficient C₂ substrates to support for the utilization by the RHA1 cells. Indeed, this condition witnessed a rapid RHA1 growth in the integrated system, as indicated by the increase in OD600 from 0.3 to 2.2. Moreover, the lipid accumulation reached 562.1 mg/L within 42 hours, equivalent to a lipid productivity rate of 321.1 mg/L/day, comparable to the lipid production rates reported in many studies on high lipid-producing algae. These results demonstrated an efficient SSF performance of the EBF system, highlighting the effectiveness of the EBF system for converting CO₂ to support the expansion of microbial cell factories.

In contrast to the SSF mode for achieving RHA1 population expansion, the WCC mode was specifically designed to enhance lipid production from the RHA1 microbial cell factories in the EBF system. Specifically, a relatively high cell density of RHA1 strains was inoculated with a limited nitrogen supply to maintain a high C/N ratio during fermentation for Rhodococcus species to accumulate high lipid content in cell. The lipid production capacity of the EBF system was investigated using both the WT and AsF strains in four rounds of WCC fermentation. The results showed that when a biomass load of 4.0 g/L of WT RHA1 cells was inoculated in the EBF system, a lipid production of 820.0 mg/L from CO₂ was achieved. Similarly, when a comparable biomass load of AsF cells (3.9 g/L) was inoculated, the lipid titer reached 1029.6 mg/L. Increasing the biomass load of the WT strain to 4.9 g/L resulted in a lipid titer of 750.4 mg/L, while inoculating 5.2 g/L of AsF cells into the EBF system achieved a higher lipid production of 1360.0 mg/L. These four routes of WCC fermentation demonstrated the reliability of EBF system for lipid production from CO₂. Moreover, the lipid productivity of the EBF system with the AsF strain reached an average number of 1194.8 mg/L/day, which is comparable to the top two recorded lipid productivity from algae Chaetoceros gracilis and Tetraselmis tetrathele. These results highlighted the potential of the EBF system for rapid lipid production from CO₂.

To further evaluate the energy conversion efficiency of the EBF system, an analysis considering three stages were conducted: solar to electricity, electricity to soluble carbon sources, and soluble carbon sources to lipid. For the stage 1 conversion the efficiency was estimated using maturing photovoltaic technology, which can achieve an efficiency of 25%. Second, in calculating the energy efficiency of the electrocatalysis stage, considering the gaseous products such as ethylene and hydrogen from electrocatalysis were not utilized by the bioconversion the energy efficiency was calculated by dividing the energy content of all soluble products (energy output) with the electricity energy input used to produce these soluble products. The analysis revealed an energy efficiency of 45.6% for the conversion of electricity into soluble products. Third, the energy efficiency of bioconversion was calculated by dividing the energy content of the produced lipid by the total energy of consumed soluble products, resulting in an average efficiency of 36.5% for the AsF strain under WCC mode. Overall, the EBF system achieved an overall energy efficiency of 4.2% for converting CO₂ to lipid.

While aspects of the disclosure have been shown and described, modifications thereof can be made without departing from the spirit and teachings of the presently disclosed subject matter. The aspects and examples described herein are exemplary only, and are not intended to be limiting. Many variations and modifications of the subject matter disclosed herein are possible and are within the scope of the present disclosure.

At least one aspect is disclosed and variations, combinations, and/or modifications of the aspect(s) and/or features of the aspect(s) made by a person having ordinary skill in the art are within the scope of the disclosure. Alternative aspects that result from combining, integrating, and/or omitting features of the aspect(s) are also within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, 5, 6 . . . ; greater than 0.10 includes 0.11, 0.12, 0.13, 0.14, 0.15, . . . ). For example, whenever a numerical range with a lower limit, RI, and an upper limit, Ru, is disclosed, any number falling within the range is specifically disclosed. In particular, the following numbers within the range are specifically disclosed: R=RI+k* (Ru-RI), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent . . . 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim means that the element is required, or alternatively, the element is not required, both alternatives being within the scope of the claim. Use of broader terms such as comprises, includes, and having should be understood to provide support for narrower terms such as consisting of, consisting essentially of, and comprised substantially of.

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the detailed description of the presently disclosed subject matter.

1. A method for the production of value added chemicals, the method comprising:
introducing carbon dioxide to a system comprising an electrochemical cell comprising a carbon dioxide reducing catalyst and an electrolyte under conditions suitable for conversion of carbon dioxide to one or more C₂ compounds;
introducing at least a portion of the one or more C₂ compounds to a second system comprising a microbe and a media under conditions suitable for conversion of the C₂ compounds to one or more C₂₊ₙ compounds where n is from about 1 to about 100.

2. The method of claim 1, wherein the one or more C₂ compounds comprise ethylene (C₂H₄), ethanol (CH₃CH₂OH), acetate (C₂H₂O₂⁻), ethylene (C₂H₄) or combinations thereof.

3. The method of claim 1, wherein the C₂ compound comprises ethanol, acetate or combinations thereof.

4. The method of claim 1, wherein the catalyst comprises Ag, Au, Cu, Zn or combinations thereof.

5. The method of claim 1, wherein the catalyst comprises a support material.

6. The method of claim 1, wherein the support material is porous.

7. The method of claim 1, wherein the catalyst comprises a supported copper catalyst.

8. The method of claim 1, wherein the catalyst comprises a nanoporous Cu-M catalyst M is a metal selected from the group consisting of Pt, Ir, Pd, Ag, Au, Rh, Ru, Zn, Sn, Ni, Fe, Re, Ga, In, Cd, TI, Ti, and combinations thereof.

9. The method of claim 1, wherein the media and electrolyte are compositionally the same.

10. The method of claim 1, wherein the microbe is a wildtype strain.

11. The method of claim 1, wherein the microbe is a mutant strain.

12. The method of claim 1, wherein the one or more C₂₊ₙ compounds is selected from the group consisting of polymers, fuels, lipids, light olefins, gluconic acid, lactic acid, malonic acid, propionic acid, triacids, citric acid, aconitic acid, xylonic acid, acetoin, furfural, levoglucosan, lysine, serine, threonine, 1,4 succinic, fumaric acid, malic acid, 2,5 furan dicarboxylic acid, 3-hydroxy propionic acid, aspartic acid, glucaric acid, glutamic acid, itaconic acid levulinic acid, 3-hydroxybutyrolactone, glycerol, sorbitol, xylitol, arabinitol, 5-hydroxymethylfurfural, ferulic acid, terpenoids, polyketides, carotenoids, polyphenols, alkaloids and combinations thereof.

13. The method of claim 1, wherein the one or more C₂₊ₙ compounds comprise lipids.

14. A method of preparing value added chemicals, the method comprising:
a means for converting carbon dioxide to one or more C₂ compounds and
a microbe wherein the C₂ compound is contacted with the microbe under conditions suitable for the formation of one or more C₂₊ₙ compounds.

15. The method of claim 14, wherein the means for converting carbon dioxide to one or more C₂ compounds is an electrochemical cell comprising a nanoporous Cu-M catalyst where M is a metal selected from the group consisting of Pt, Ir, Pd, Ag, Au, Rh, Ru, Zn, Sn, Ni, Fe, Re, Ga, In, Cd, TI, Ti and combinations.

16. The method of claim 14, wherein the C₂ compound comprises ethanol, acetate or combinations thereof.

17. The method of claim 14, wherein the microbe comprises a bacteria.

18. The method of claim 14, wherein the microbe is mutated.

19. The method of claim 14, wherein the one or more C₂₊ₙ compounds is selected from the group consisting of polymers, fuels, lipids, light olefins, gluconic acid, lactic acid, malonic acid, propionic acid, triacids, citric acid, aconitic acid, xylonic acid, acetoin, furfural, levoglucosan, lysine, serine, threonine, 1,4 succinic, fumaric acid, malic acid, 2,5 furan dicarboxylic acid, 3-hydroxy propionic acid, aspartic acid, glucaric acid, glutamic acid, itaconic acid levulinic acid, 3-hydroxybutyrolactone, glycerol, sorbitol, xylitol, arabinitol, 5-hydroxymethylfurfural, ferulic acid, terpenoids, polyketides, carotenoids, polyphenols, alkaloids and combinations thereof.

20. The method of claim 14, wherein the one or more C₂₊ₙ compounds comprise lipids.
